(19) United States
(12) Patent Application Publication
(10) Pub. No.: US 2021/0398635 A1
(43) Pub. Date: Dec. 23, 2021

(54) MEDICATION COMPLIANCE DEVICE

(71) Applicant: Metab Faleh Alwethairi ALANAZI, Riyadh (SA)

(72) Inventor: Metab Faleh Alwethairi ALANAZI, Riyadh (SA)

(21) Appl. No.: 17/110,936

(22) Filed: Dec. 3, 2020

(30) Foreign Application Priority Data
Jun. 22, 2020 (SA) 120410801

(51) Int. Cl. G16H 20/13; G16H 40/67; G16H 80/00; A61J 7/02

(52) U.S. Cl. CPC G16H 20/13; G16H 40/67; G16H 80/00; A61J 2200/74; A61J 2200/30; A61J 2200/72; A61J 7/02

(57) ABSTRACT

The invention relates to a portable drug dispenser system that includes a chamber for holding a plurality of separately contained medication canisters, a dispensing mechanism for dispensing one or more of the plurality of medication products from the medication canisters, and includes a processor to determine the time of each activation of the dispensing mechanism and to transmit the medication to a locked dispensing station which is only openable by either a password or biornetric means. The dispenser also includes a connection to a smart device to provide the user information and alarms.

### COPYRIGHT STATEMENT

Trademarks are used in the disclosure of the invention and the applicants make no claim to any trademarks referenced.

### CROSS-REFERENCE TO RELATED APPLICATION

This application claims priority from Kingdom of Saudi Arabia Patent Application No. 120410801, filed Jun. 22, 2020, titled Medication compliances which is hereby incorporated by reference herein for all purposes.

### BACKGROUND OF THE INVENTION

### 1) Field of the Invention

The invention relates to portable drug dispensing devices and compliance system, and, more particularly to portable drug dispensers having one or more pill magazines with one or more pill canisters for holding one or more drugs, a dispensing mechanism for dispensing the drugs, and a processor/control system for determining the time of activation of the dispensing mechanism and to communicate with remote smart devices such as a computer of smart phone or tablet.

### 2) Description of Related Art

Drug related problems are any event or circumstance involving drug treatment (pharmacotherapy) that interferes with the optimal delivery of medical care. This includes clinical problems related to the use of medications or “drugs” in the field of pharmaceutical care.

The art defines seven categories of Drug Related problems:

1) Unnecessary drug therapy. This occurs when the patient is taking too many medications for their condition and the drug is simply not needed.

2) Wrong drug. This occurs when a patient is taking medication that does not treat his condition. Ex. A heart medication to treat an infection.

3) Dose is too low. This occurs when a patient is taking medication that is not strong enough to get beneficial or therapeutic effects.

4) Dose too high. This occurs when a patient is given medication that is too strong and is causing detrimental effects or is simply not necessary.

5) Adverse drug reaction. This occurs when a patient has an allergic response to a medication.

6) Inappropriate adherence. This occurs when a patient chooses not to or forgets to take a medication.

7) Needs additional drug therapy. This occurs when a patient needs more medication to treat their condition.

In the course of clinical practice, drug related problems are often identified, prevented, and/or resolved by pharmacists in the course of medication therapy management, as experts on the safety and efficacy of medications. However, other healthcare professionals may also respond and manage drug related problems.

For many patients, medications need to be tightly controlled. For example, if the medications include restricted or controlled medicines.

Moreover, there are many incidents of suicide by prescription drug overdose, medication theft, accidental misuse of medications, and the un-authorized use of someone else's medication.

Therefore, automatic medication dispensers are needed to dispense medicines and help prevent the issues related to drug related problems.

BRIEF SUMMARY OF THE INVENTION

The invention in one form is directed to automated drug dispensing at home which includes prescription capture, prescription compliance, tablet counting, tablet dispensing, tablet accounting, prescription storage monitoring and prescription security.

The invention in another form alerts the patient when it is time to take their medication and dispenses the medication only when the patient confirms that they are going to take it.

In yet another form the invention provides a means for the pharmacist and physician to monitor compliance by providing reports and information as to times of medication dispensing, amount of medication used and amount of medication unused.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 is an overall system block diagram;

FIG. 2 is a system diagram;

FIG. 3 is a flow chart of the system functions starting a when the patient receives the system to when the patient takes the system home;

FIG. 4 is a flow chart of the system functions starting at the system alarming so the patient is alerted to take their medications to the system dispensing and accounting prior to the next dose;

FIG. 5 is a flow chart of the system environmental monitoring;

FIG. 6 is a system diagram showing the elements of the invention connected to the control system.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

the following description, numerous specific details are set forth.

However, it is understood that embodiments of the invention may be practiced without these specific details. In other instances, well-known structures and techniques have not been shown in detail in order not to obscure the understanding of this description. Those of ordinary skill in the art, with the included descriptions, will be able to implement appropriate functionality without undue experimentation.

References in the specification to “one embodiment” or “an embodiment,” may indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that such feature, structure, or characteristic may be deployed in connection with other embodiments whether or not explicitly described.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms people, client, user, patient, consumer and individual are used interchangeably to mean an individual who uses the invention.

The term platform, system, process and method are used interchangeably to mean a process of the invention.

All the input and display fields shown in the detailed description of the invention are optional and some embodiments may use all or only some of the input fields.

As used in the invention the term prescription is intended to refer to the prescription for medication tablets or capsules.

The term physician as used in this specification is intended to mean the prescribing entity.

The term pharmacist as used in this specification is intended to mean the entity filing the system.

The term smart device as used in this specification is intended to mean a mobile smart device such as a phone or tablet which is connected to the system via wireless communication.

The term computer as used in this specification is intended to mean a computing device which can be connected to the system either via wireless communication or wired communication.

The prior art does not provide for a personal system that provides an automated dispensing of medication and connectivity to remote computer systems and smart devices. U.S. Patent Application 20170326034A1, Publication Date: Nov. 16, 2017 the contents of which are incorporated by reference in its entirety describes a typical dispensing system.

As discussed previously, Drug related problems, (DRPs) is any event or circumstance involving drug treatment (pharmacotherapy) that interferes with the optimal delivery of medical care. DRPs represent the categorization and definition of clinical problems related to the use of medications or “drugs” in the field of pharmaceutical care. In the course of clinical practice, DRPs are often identified, prevented, and/or resolved by pharmacists in the course of medication therapy management, as experts on the safety and efficacy of medications, but other healthcare professionals may also manage DRPs. DRPs can be categorized as Unnecessary drug therapy, Wrong drug, Dose is too low, Dose too high, Adverse drug reaction, Inappropriate adherence, Needs additional drug therapy.

Medical professionals also provide further break down with respect to

DRPs into the following categories:

Indication,
a. Requires Additional drug therapy:
   i. Untreated condition
   ii. Preventative/prophylactic
   iii. Synergistic potentiating
b. Unnecessary drug therapy
   i. No medical indication
   ii. Duplicate therapy
   iii. Non-drug therapy indicated
   iv. Treating avoidable adverse drug reactions (ADR)

Effectiveness
a. Requires different drug product
   i. More effective drug available
   ii. Condition refractory to drug
   iii. Dosage form is inappropriate
   iv. Not effective for condition
b. Dosage too low
   i. Wrong dose
   ii. Frequency inappropriate
   iii. Duration inappropriate
   iv. Drug interaction

Safety
a. Adverse Drug Reaction
   i. Undesirable effect
   ii. Unsafe drug for patient
   iii. Dose changed too quickly
   iv. Allergic reaction
   v. Contraindications present
b. Dosage too high
   i. Wrong dose
   ii. Frequency inappropriate
   iii. Incorrect administration
   iv. Drug interaction

Compliance or Adherence
a. Non-adherence
   i. Directions not understood
   ii. Patient prefers not to take
   iii. Patient forgets to take
   iv. Drug product too expensive
   v. Cannot swallow/administer
   vi. Drug product not available

All above mentioned Drug Related Problems have consequences and costs related to them and can severely affect the outcomes of the patient therapy. The consequences include death, increase clinic visits, admission to hospitals, failure of the liver or kidney, pain, illness, toxicity caused by drugs and drug interactions, untreated conditions, wasted resources, Increased hospital bed occupancy, increased hospital and emergence department visits, failure of therapy, loss of jobs and increased cost.

There are also hidden costs in the United States in that two out of three dispensed medications were unused, with national projected costs ranging from 82.48 to 85.4B. This wastage raises concerns about adherence, cost and safety; additionally, it points to the need for public awareness and policy to reduce wastage. The most frequent cause of disposal or unuse were disease/condition improved (42.4%), forgetfulness (5.8%) and side effects (6.5%) were reasons cited for their nonuse. “Throwing medications in the trash” was found being the common method of disposal (63%). From the environmental viewpoint, wasted medications adversely affect humans, animals, and plants if they are not safely disposed of.

Research has shown that pain medications (23.3%) and antibiotics (18%) were most commonly reported as unused, whereas the investigators found that 17% of medications for chronic conditions (hypertension, diabetes, cholesterol, heart disease) and 8.3% for mental health problems were commonly reported as unused. The participants in the study indicated that the pharmacy is the preferred location for drug disposal. The combination of unnecessary prescriptions, together with low medication adherence, silently contributes to the waste of finite resources, which further accentuates the issues related to the already rising healthcare costs.

Therefore, there is an unmet need to control the distribution of medication and provide a feed back loop where the pharmacist can confirm that the patient has used their medication and provide feed back to the patient and the physician.

The solution to these problems includes the instant invention which promotes patient counseling, health informatics and the use of artificial intelligence.

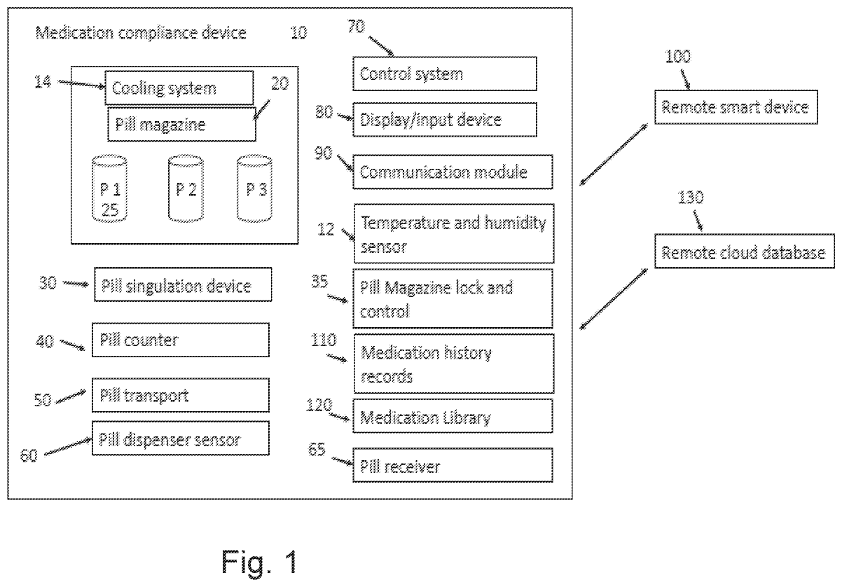

Referring now to the FIG. 1-6, and more particularly to FIG. 1, there is shown an overall system block diagram of the medication compliance device 10 for home use by a patient having pill magazine 20 and pill canisters 25. The design anticipates multiple magazines 20 and each magazine 20 contains 1-8 pill canisters 25. The pill canister 25 is in communication with a pill singulation device 30 which is capable of transporting the pill in a pill transport 50 device which transporting the pill to the pill dispenser 55 which is monitored by a pill dispenser sensor 60 and said pill is deposited in a pill receiver 65.

The control system 70 has a display/input 80 which allows the pharmacist and the physician to enter information into the medication compliance device 10. The display/input 80 can be a touch screen or a separate display and input device such as a keyboard, The control system 70 is in communication with the communication module 90 which facilitates communication with the remote cloud database 130 and the remote smart device 100.

The temperature and humidity sensor communicates with the control system 70 to provide enviromental information to the control system so that it can display alerts on the display/input device 80 and the control system 70 initiates the cooling and humidity control system 14. The control system 70 also controls the pill magazine lock 35 and control module 70 uses the lock 35 to limit the access to the pharmacist. The control system 70 also controls the access to the medication history records 110 and medication library 120. The medication compliance device 10 communicates with the remote smart device 100 to provide updates and information including alarms, medication status, communications with the physician and pharmacist.

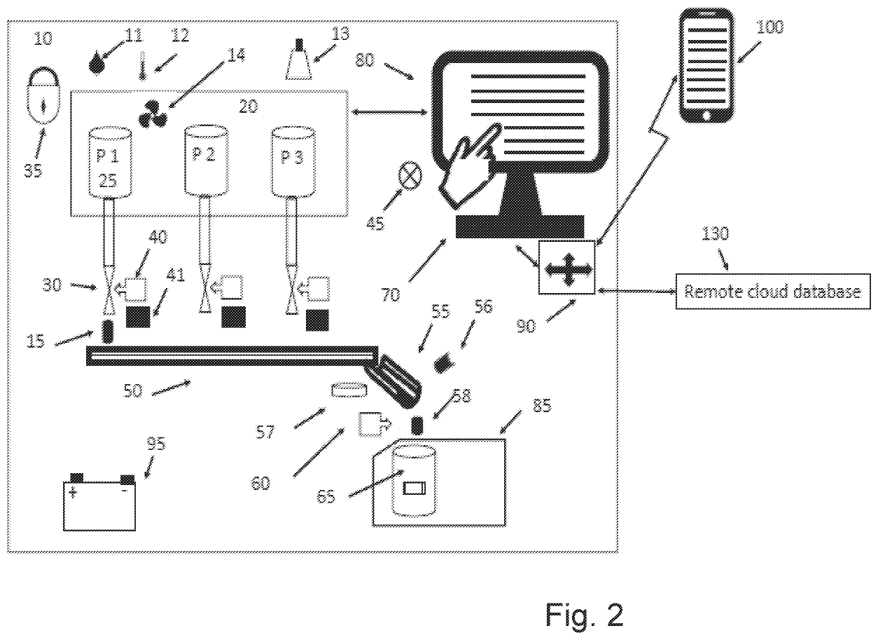

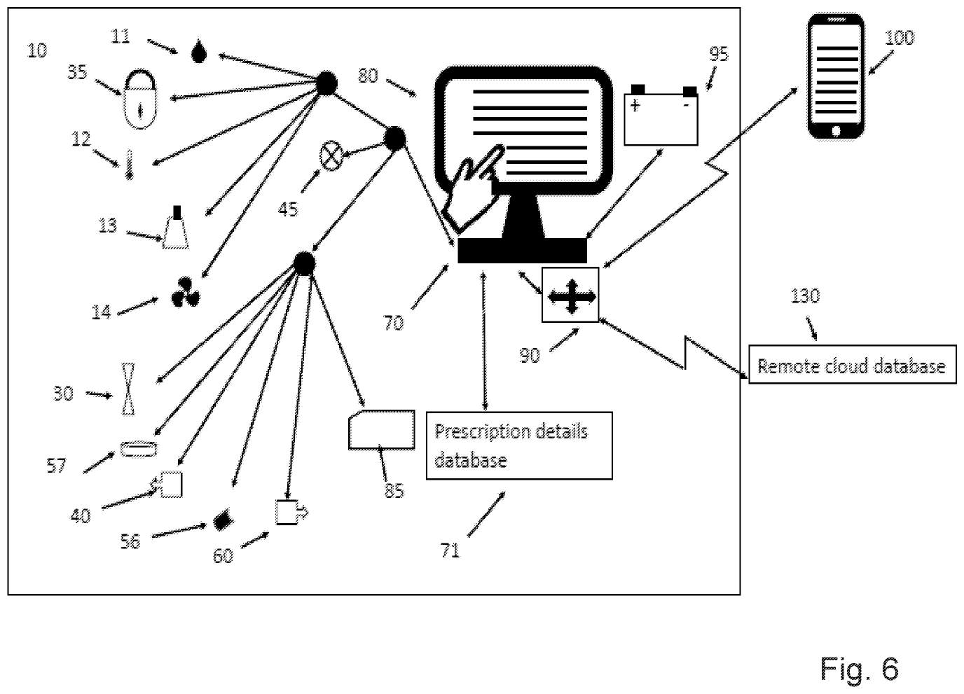

Referring to FIG. 2 which is a system diagram, there is shown the medication compliance device 10 having pill magazine 20 and pill canisters 25. When the pharmacist initiates the medication compliance device 10 using the display/input device 80 and the proper password, the pharmacist can also set limits or set points for humidity and temperature as well as patient information, medication information, amounts of medication loaded into the medication compliance device 10 and the medication refill information. This information is placed in the prescription details database 71 (as shown in FIG. 6) on the control system 70, all prescription information and the refill thresholds are inputted for each prescription. The prescription details database 71 can optionally be either on the control system 70 or on the remote cloud database 130.

The medication compliance device 10 also has a temperature 12 and humidity sensor 11, temperature and humidity control system 14 and alarm 13. The temperature 12 and humidity sensor 11 are in communication with the control system 70 which is in communication with the temperature and humidity control system 14 and alarm 13. If the temperature or humidity is outside of the present limits the control system 70 engages the temperature and humidity control system 14 and alarm 13. As shown the design anticipates multiple magazines 20 and each magazine 20 containing 1-8 pill canisters 25. Each canister has a unique number which is associated by the medication compliance device 10 to the specific canister 25. The pharmacist can access the magazine 20 by using the lock medication compliance device pill magazine pill magazine lock 35 which is controlled by a password entered into the display/input 80 of the control system 70. When the pharmacist enters the appropriate password then the control system 70 unlocks the pill magazine 20 using lock 35. The lock 35 is in communication with the control system 70 and the control system 70 can communicate electronically via wire connection or wirelessly with the lock 35, The wireless connection can be any suitable wireless technology such as Low Power Wide Area Networks (LPWANs) such as Bluetooth, Bluetooth low energy, WiFi, Z-Wave, Thread and Zigbee. When the medication compliance device 10 is enabled to dispense a pill, the pill magazine 20 which is in communication with a pill singulation device 30 dispenses a single pill 15 which is counted by the pill counter system 41 and sensor 40. The pill 15 is then transported using the pill transport device 50 to the pill dispenser 55 which is monitored by a pill dispenser sensor 60. The pill transport device 50 can be any mechanism enabled to transport pills or capsules including but not limited to a belt conveyor, pressurized tube or screw conveyor. Once the pill enters the pill dispenser 55 the pill dispenser sensor 60 records the dispensing and the pill 15 becomes pill 58 which is a dispensed pill. Pill dispenser 55 can also be enabled by artificial intelligence and can be equipped with a scale 57 which measures the weight of the pill 58 which can then be transferred to the control system 70 and compared to the record in the Medication Library 120 prescription details database 71 (as shown in FIG. 6) and used as a quality control means to ensure that the pill dispensed is the correct pill that has been directed to be dispensed by the control system. Alternatively, a camera system 56 could also be attached to the pill dispenser 55 and a picture of the dispensed pill could be transferred to the control system 70 and compared to the record in the Medication Library 120 prescription details database 71 to ensure that the pill dispensed is the correct pill that has been directed to be dispensed by the control system.

The control system 70 has a display/input 80 which allows the pharmacist and the physician to enter information into the medication compliance device 10. The control system 70 is in communication with the communication module 90 which facilitates communication with the remote cloud database 130 and the remote smart device 100. The communication module can use any suitable wireless technology such as cellular which can include but is not limited to Global System for Mobile communication, Code Division Multiple Access, Wireless in Local Loop, General Packet Radio Services or it can also utilize Low Power Wide Area Networks (LPWANs) such as Bluetooth, Bluetooth low energy, WiFi, Z-Wave, Thread and Zigbee.

The temperature and humidity sensor communicates with the control system 70 to provide environmental information to the control system so that it can display alerts on the display/input device 80 or on remote smart device 100. The control system 70 also controls the pill magazine lock 35 and control system 70 which limits the access to the pharmacist. The control system 70 also controls the access to the medical history records 110 and medical library 120. The medical history records 110 can contain any information that the pharmacist or physician deems necessary for the care of the patient.

The system is powered by power supply 95 which can be a battery, rechargeable battery, or a power supply connected to line current such as a 110 volt or 220 volt supply.

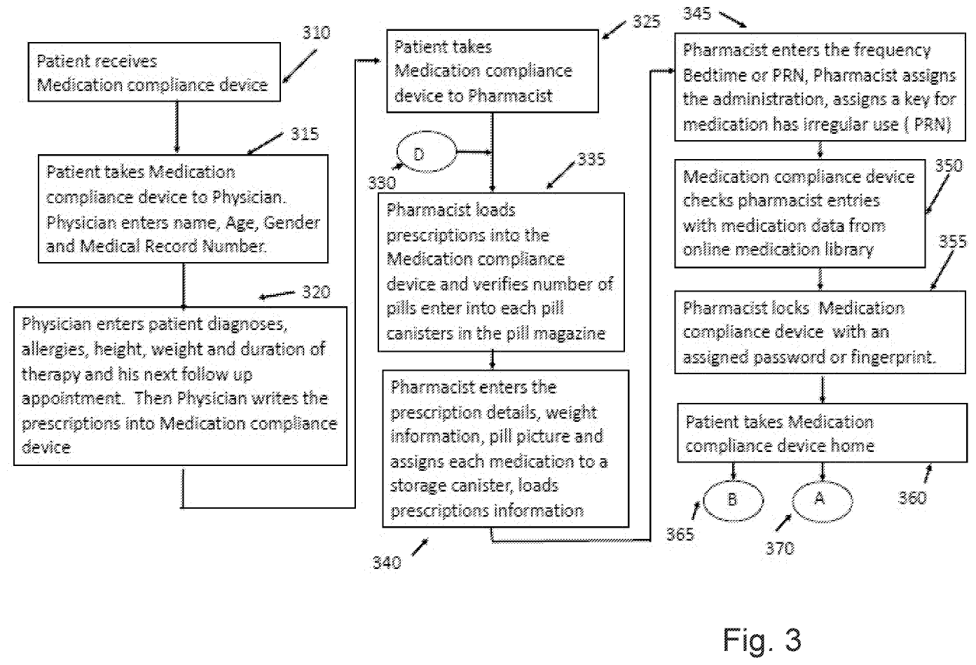

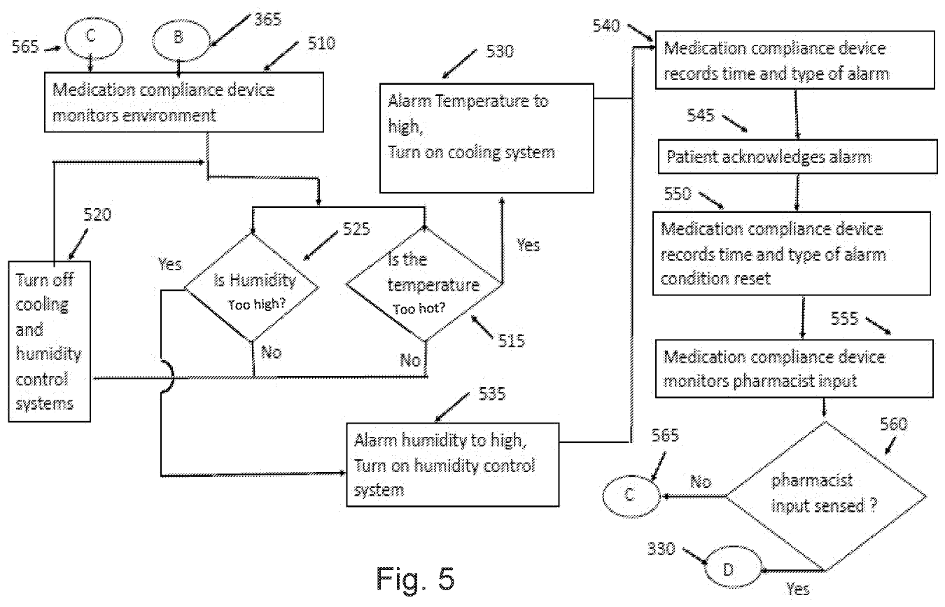

Referring to the flowchart of FIG. 3 which shows the system functions starting when the patient receives the system to when the patient takes the system home. Patient receives medication compliance device in step 310 and the patient takes the medication compliance device 10 to the physician in step 315. The physician enters name, age, gender and Medical Record Number, medical insurance number, medical number or Medicaid number into the medication compliance device 10. Next in step 320 the physician enters patient diagnoses, allergies, height, weight and duration of therapy and his next follow up appointment. Then physician writes the prescriptions into medication compliance device 10 and they are stored in the control system 70 prescription details database 71 or they can be optionally stored on the remote cloud database 130. The patient takes medication compliance device to the pharmacist step 325, Control transfer “D” step 330 is a transfer of control from the flow chart in FIG. 5. Next in step 335 the pharmacist loads prescriptions into the medication compliance device 10 using display/input 80 and verifies number of pills enter into each pill canister 25 in the pill magazine 20. The pharmacist filling process for pill canisters 25 provides a means for the pharmacist to assign each drug to a specific canister, by canister number, drug name and number of pills or capsules assigned to canister 25. In step 340 the pharmacist enters the prescription details into the medication compliance device 10 using display/input 80, assigns each medication to a storage canister 25, loads pill weight information, pill picture, prescriptions information into the prescription details database 71 on the medication compliance device 10 using display/input 80. The prescription details database 71 can optionally be either on the control system 70 or on the remote cloud database 130.

In step 345 the pharmacist enters the frequency such as take at bedtime or PRN, pharmacist assigns the administration, assigns a key for medication that has an irregular use which usually means that the patient takes the prescription as needed (PRN—stands for ‘pro re rata,’ which means that the administration of medication is not scheduled, instead, the prescription is taken as needed) into the medication compliance device 10 the scheduling function which is part of the control system 70 using display/input device 80. In step 350 the medication compliance device 10 checks pharmacist entries with medication data from online medication library 120, prescription details database 71 and medical history records 110. In step 355 the pharmacist locks the medication compliance device 10 with an assigned password or fingerprint. In step 360 the patient takes medication compliance device 10 home.

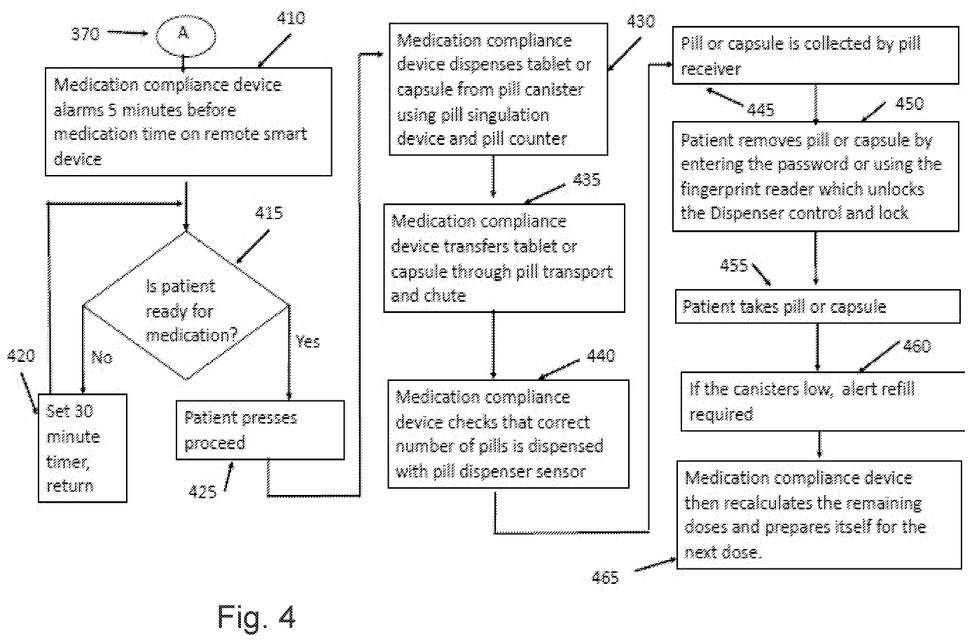

Control transfer “A” step 370 is transfer of control from the flow chart in FIG. 4.

Control transfer “B” step 365 is a transfer of control from the flow chart in

FIG. 5.

Referring to the flowchart of FIG. 4 which shows the system functions starting at the system alarming so the patient is alerted to take their medications to the system dispensing and accounting prior to the next dose. The medication compliance device 10 transfers control from step 370. In step 410 the medication compliance device 10 scheduling function which is part of the control system 70 alarms 5 minutes before the medication time on remote smart device 100. The medication time is associated within the control system with a medication canister 25 and specific medication contained in the medication canister 25. In step 415 the control system 70 determines if the patient is ready to take the medication; if they are not the system sets a timer for 30 minutes in step 420 and test if the patient is ready to take the medication after the timer has expired. If the patient is ready and presses the proceed with medication dispensing key step 425 then the control system 70 instructs the medication compliance device 10 to begin the process to dispense the required medication contained in the specific medication canister 25 that contains the required medication. The dispensing of the medication (pill or capsule) from the canister 25 uses a pill singulation device 30 to deposit the medication into the pill transport device 50 and chute 55 step 435. In step 440 the medication compliance device 10 checks that correct number of pills has been dispensed with pill dispenser sensor 60. The dispensed pill 58 can undergo a quality control step incorporated into the medication compliance device 10. The chute 55 can be equipped with a scale 57 which measures the weight of the pill which can then be transferred to the control system 70 and used as a quality control means to ensure that the pill dispensed is the correct pill that has been directed to be dispensed by the control system. The control system 70 uses the measured weight and the weight information in the Medication Library 120 and prescription details database 71 to verify that the pill 58 dispensed is the correct pill. If the pill 58 is not the correct pill then the medication compliance device 10 sets alarm 13 and sends an alarm to the remote smart device 100. Alternatively, a camera system 56 could also be attached to the pill dispenser 55 and a picture of the dispensed pill could be transferred to the control system 70 and compared to the record in the Medication Library 120 and prescription details database 71 to ensure that the pill 58 dispensed is the correct pill that has been directed to be dispensed by the control system. If the pill 58 is not the correct pill then the medication compliance device 10 sets alarm 13 and sends an alarm to the remote smart device 100. In step 445 the pill or capsule is collected by pill receiver where it is located for the patient to withdraw the pill or capsule 58 from the medication compliance device 10. In step 450 the patient removes pill or capsule by entering the password or using the fingerprint reader 45 which unlocks the dispenser control and lock 85 so that the patient can take the pill or capsule 58 in step 455. If the canister 25 is low then the medication compliance device 10 alerts the patient that a refill is need by sending the alert to the remote smart device 100. The refill threshold is inputted by the pharmacist when they set up the medication compliance device 10. In step 465 the medication compliance device 10 using control system 70 recalculates the remaining doses and prepares itself for the next dose.

Referring to the flowchart of FIG. 5 which shows the system environmental monitoring functions. The medication compliance device 10 transfers control from step 365 or step 565. In step 510 the medication compliance device 10 monitors the environment in the pill magazine 20 and it checks to determine if the temperature is too hot in step 515 and if the humidity is too high in step 525. If the temperature is too hot, then the cooling system is turned on in step 530 and if the humidity is too high the humidity control system is turned on in step 535. Otherwise the cooling and humidity control systems are turned off in step 520. The humidity and temperature limits are entered by the pharmacist at the setup of the medication compliance device 10.

In step 540 the medication compliance device 10 records time and type of alarm and when the patient acknowledges the alarm in step 545 using display/input device 80 to input the information into the control system 70. This is just one of the many logs which the medication compliance device 10 keeps. The logs include temperature, humidity, dispensing records including time, number of pills or capsules dispensed, number of delay timers per prescription initiated, pill and capsule dispensed by medication type per day, month and year, alarm type and time and date of alarm.

In step 550 the medication compliance device 10 records time and type of alarm condition that is reset. In step 555 the medication compliance device 10 monitors pharmacist input. If the pharmacist input is sensed in step 560 then control is transferred to 330 and if not then the Medication compliance device 10 transfers control to step 565.

As shown in FIG. 6 the control system 70 is In communication wirelessly with the remote cloud database 130 and the remote smart device 100. It is also in communication with the prescription details database 71, temperature 12, humidity sensor 11, alarm 13, pill singulation device 30, medication compliance device pill magazine lock 35, pill counter 40, fingerprint reader 45, pill dispenser sensor 60, display/input 80, dispenser control and lock 85, communication module 90, temperature and humidity control device 14, scale 57 and camera 56. The prescription details database 71 can optionally be either on the control system 70 or on the remote cloud database 130.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A medication compliance device for home use by a patient for dispensing at least one pill, comprising of:
a power supply in communication with a control system and said control system in communication with a display and input device;
said control system in communication with a remote smart device;
said control system in communication with a prescription detail database;
said control system in communication with a remote cloud database;
said control system accessible by a physician by using a password;
said control system accessible by a pharmacist by using a password;
said control system in communication with a pill magazine, a pill singulation device, a pill counter, a pill transport device, a pill dispenser sensor, a temperature sensor, a humidity sensor, a cooling and humidity control system, an alarm, a medication compliance device lock, a fingerprint reader, a communication module;
said pill magazine containing at least one pill canister;
said pill canister is in communication with said pill singulation device;
said pill singulation device capable of depositing said at least one pill in said pill transport device;
said at least one pill capable of being transported by said transport device to a pill dispenser which is monitored by a pill dispenser sensor to verify that said at least one pill is dispensed; and
said pill is deposited in said pill receiver when said at least one pill is dispensed.

2. The pill dispenser of claim 1, wherein said pill dispenser has a scale to weigh said at least one pill and said scale is in communication with said control system which is in communication with said communication module which is in communication with said remote cloud database and said remote cloud database having the weight of said at least one pill contained in said prescription details database on said remote cloud database and said control system comparing said weight of said at least one pill with the weight of said at least one pill from said prescription details database on said remote cloud database to determine if the pill dispense is the correct pill.

3. The pill dispenser of claim 1, wherein said pill dispenser has a scale to weigh said at least one pill and said scale is in communication with said control system which is in communication with said prescription details database on said control system and said prescription details database having the weight of said at least one pill and said control system comparing said weight of said at least one pill with the weight of said at least one pill from said prescription details database to determine if the pill dispense are the correct pill.

4. The pill dispenser of claim 1, wherein said pill dispenser has a camera to take a picture of at least one pill and said camera is in communication with said control system which is in communication with said communication module which is in communication with said remote cloud database and said remote cloud database having the picture of said at least one pill contained in said prescription details database on said remote cloud database and said control system comparing said picture of said at least one pill with the picture of said at least one pill from said prescription details database on said remote cloud database to determine if the pill dispense is the correct pill.

5. The pill dispenser of claim 1, wherein said pill dispenser has a camera to take a picture of at least one pill and said camera is in communication with said control system which is in communication with said prescription details database on said control system and said prescription details database having the picture of said at least one pill and said control system comparing said picture of said at least one pill with the picture of said at least one pill from said prescription details database to determine if the pill dispense is the correct pill.

6. The pill dispenser of claim 1, wherein said control system in communication with a temperature sensor and said control system is in communication with said cooling and humidity control system and said alarm and said communication system displays said alarm when said temperature sensor reaches a set point entered in said prescription details database.

7. The alarm of claim 6, wherein the alarm is displayed on a remote smart device.

8. The pill dispenser of claim 1, wherein said control system in communication with a humidity sensor and said control system is in communication with said cooling and humidity control system and said alarm and said communication system displays said alarm when said humidity sensor reaches a set point entered in said prescription details database.

9. The alarm of claim 8, wherein the alarm is displayed on a remote smart device.

10. The pill dispenser of claim 1, wherein said control system in communication with a remote smart device to provide the patient with updates, information, alarms, medication status, communications with said physician and communications with said pharmacist.

11. The medication compliance device of claim 1, wherein the said physician by using a password enters prescription information into said prescription details database on said control system.

12. The medication compliance device of claim 1, wherein the said pharmacist by using a password enters prescription information into said prescription details database on said control system.

13. A medication compliance device for home use by a patient for dispensing at least one pill, comprising of:
a power supply in communication with a control system and said control system in communication with a display and input device;
said control system accessible by a physician by using a password;
said control system accessible by a pharmacist by using a password;
said control system in communication with a wireless communication module and said wireless communication module in communication with a remote smart device;
said control system in communication with a wireless communication module and said wireless communication module in communication with a remote cloud database;
said control system in communication with a pill singulation device;
said control system in communication with a pill counter sensor;
said control system in communication with a pill dispenser sensor;
said control system in communication with a temperature sensor;
said control system in communication with a humidity sensor;
said control system in communication with a cooling and humidity control system; said control system in communication with an alarm;
said control system in communication with a medication compliance device lock;
said control system in communication with a fingerprint reader;
said control system in communication with a scale;
said control system in communication with a camera;
said control system in communication with a dispenser control and lock;
said pill singulation device communication with at least one pill canister.

14. The pill dispenser of claim 13, wherein said control system is in communication with said scale to weigh said at least one pill and said control system is in communication with said communication module which is in communication with said remote cloud database and said remote cloud database having the weight of said at least one pill contained in said prescription details database on said remote cloud database and said control system comparing said weight of said at least one pill with the weight of said at least one pill from said prescription details database on said remote cloud database to determine if the pill dispensed is the correct pill.

15. The pill dispenser of claim 13, wherein said control system is in communication with said scale to weigh said at least one pill and said control system which is in communication with said prescription details database on said control system and said prescription details database having the weight of said at least one pill and said control system comparing said weight of said at least one pill with the weight of said at least one pill from said prescription details database to determine if the pill dispensed is the correct pill.

16. The pill dispenser of claim 13, wherein said control system is in communication with said camera to take a picture of at least one pill and said control system which is in communication with said communication module which is in communication with said remote cloud database and said remote cloud database having the picture of said at least one pill contained in said prescription details database on said remote cloud database and said control system comparing said picture of said at least one pill with the picture of said at least one pill from said prescription details database on said remote cloud database to determine if the pill dispensed is the correct pill.

17. The pill dispenser of claim 13, wherein said control system has a camera to take a picture of at least one pill and said control system which is in communication with said prescription details database on said control system and said prescription details database having the picture of said at least one pill and said control system comparing said picture of said at least one pill with the picture of said at least one pill from said prescription details database to determine if the pill dispensed is the correct pill.

18. The medication compliance device of claim 13, wherein the said physician by using a password enters prescription information into said prescription details database on said control system.

19. The medication compliance device of claim 13, wherein the said pharmacist by using a password enters prescription information into said prescription details database on said control system.
